SEQUENCE LISTING (15 sequences)

SEQ ID NO: 1 - protein, 5 aa, synthetic construct
SYVMH

SEQ ID NO: 2 - protein, 6 aa, synthetic construct
NPYNDG

SEQ ID NO: 3 - protein, 12 aa, synthetic construct
GTYYYGTRVFDY

SEQ ID NO: 4 - protein, 16 aa, synthetic construct
RSSKSLQNVNGNTYLY

SEQ ID NO: 5 - protein, 7 aa, synthetic construct
RMSNLNS

SEQ ID NO: 6 - protein, 9 aa, synthetic construct
MQHLEYPIT

SEQ ID NO: 7 - protein, 556 aa, Homo sapiens [sequence omitted]

SEQ ID NO: 8 - protein, 450 aa, synthetic construct
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 9 - protein, 213 aa, synthetic construct
DIQMTQSPSTLSASVGDRVTITCSASSRVGYMHWYQQKPGKAPKLLIYDTSKLASGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 121 aa, synthetic construct
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSS

SEQ ID NO: 11 - protein, 112 aa, synthetic construct
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIK

SEQ ID NO: 12 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 13 - protein, 107 aa, synthetic construct [sequence omitted]

SEQ ID NO: 14 - protein, 451 aa, synthetic construct
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 15 - protein, 219 aa, synthetic construct
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0176355 A1
(43) Pub. Date: Jun. 25, 2026

(54) COMBINATION OF ANTI CD19 ANTIBODY WITH A BCL-2 INHIBITOR AND USES THEREOF

(71) Applicant: Incyte Corporation, Wilmington, DE (US)

(72) Inventors: Jan Endell, Munich (DE); Konstantin Petropoulos, Munich (DE); Peter Kelemen, Munich (DE); Rainer Boxhammer, Aying (DE); Markus Rückert, Kottgeisering (DE)

(21) Appl. No.: 19/241,675

(22) Filed: Jun. 18, 2025

Related U.S. Application Data
(63) Continuation of application No. 16/342,645, now Pat. No. 12,358,983.

(30) Foreign Application Priority Data
Oct. 28, 2016 (EP) 16196184.2

(51) Int. Cl. C07K 16/28; A61K 9/00; A61K 31/496; A61K 39/395; A61P 35/02

(52) U.S. Cl. CPC C07K 16/2803; A61K 9/0019; A61K 31/496; A61K 39/395; A61P 35/02

(57) ABSTRACT

The present disclosure describes a pharmaceutical combination of an anti-CD19 antibody and a BCL-2 inhibitor for the treatment of non-Hodgkin's lymphoma, chronic lymphocytic leukemia and/or acute lymphoblastic leukemia.

### SEQUENCE LISTING

This application contains a Sequence Listing that has been submitted electronically as an XML file named 20443-0685002_SL_ST26.xml. The XML file, created on Jun. 13, 2025, is 16,486 bytes in size. The material in the XML file is hereby incorporated by reference in its entirety.

### FIELD OF THE INVENTION

The present disclosure is related to a pharmaceutical combination of an anti-CD19 antibody and a BCL-2 inhibitor for the treatment of non-Hodgkin's lymphoma, chronic lymphocytic leukemia and/or acute lymphoblastic leukemia.

### BACKGROUND

B cells are lymphocytes that play a large role in the humoral immune response. They are produced in the bone marrow of most mammals, and represent 5-15% of the circulating lymphoid pool. The principal function of B cells is to make antibodies against various antigens, and are an essential component of the adaptive immune system.

Because of their critical role in regulating the immune system, disregulation of B cells is associated with a variety of disorders, such as lymphomas, and leukemias. These include non-Hodgkin's lymphoma (NHL), chronic lymphocytic leukemia (CLL) and acute lymphoblastic leukemia (ALL).

NHL is a heterogeneous malignancy originating from lymphocytes. In the United States (U.S.), the incidence is estimated at 65,000/year with mortality of approximately 20,000 (American Cancer Society, 2006; and SEER Cancer Statistics Review). The disease can occur in all ages, the usual onset begins in adults over 40 years, with the incidence increasing with age. NHL is characterized by a clonal proliferation of lymphocytes that accumulate in the lymph nodes, blood, bone marrow and spleen, although any major organ may be involved. The current classification system used by pathologists and clinicians is the World Health Organization (WHO) Classification of Tumours, which organizes NHL into precursor and mature B-cell or T-cell neoplasms. The PDQ is currently dividing NHL as indolent or aggressive for entry into clinical trials. The indolent NHL group is comprised primarily of follicular subtypes, small lymphocytic lymphoma, MALT (mucosa-associated lymphoid tissue), and marginal zone; indolent encompasses approximately 50% of newly diagnosed B-cell NHL patients. Aggressive NHL includes patients with histologic diagnoses of primarily diffuse large B cell (DLBL, DLBCL, or DLCL) (40% of all newly diagnosed patients have diffuse large cell), Burkitt's, and mantle cell. The clinical course of NHL is highly variable. A major determinant of clinical course is the histologic subtype. Most indolent types of NHL are considered to be incurable disease. Patients respond initially to either chemotherapy or antibody therapy and most will relapse. Studies to date have not demonstrated an improvement in survival with early intervention. In asymptomatic patients, it is acceptable to “watch and wait” until the patient becomes symptomatic or the disease pace appears to be accelerating. Over time, the disease may transform to a more aggressive histology. The median survival is 8 to 10 years, and indolent patients often receive 3 or more treatments during the treatment phase of their disease. Initial treatment of the symptomatic indolent NHL patient historically has been combination chemotherapy. The most commonly used agents include: cyclophosphamide, vincristine and prednisone (CVP); or cyclophosphamide, adriamycin, vincristine, prednisone (CHOP). Approximately 70% to 80% of patients will respond to their initial chemotherapy, duration of remissions last on the order of 2-3 years. Ultimately, the majority of patients relapse. The discovery and clinical use of the anti-CD20 antibody, rituximab, has provided significant improvements in response and survival rate. The current standard of care for most patients is rituximab+CHOP (R-CHOP) or rituximab+CVP (R-CVP). Interferon is approved for initial treatment of NHL in combination with alkylating agents, but has limited use in the U.S. Rituximab therapy has been shown to be efficacious in several types of NHL, and is currently approved as a first line treatment for both indolent (follicular lymphoma) and aggressive NHL (diffuse large B cell lymphoma). However, there are significant limitations of anti-CD20 monoclonal antibody (mAb), including primary resistance (50% response in relapsed indolent patients), acquired resistance (50% response rate upon re-treatment), rare complete response (2% complete resonse rate in relapsed population), and a continued pattern of relapse. Finally, many B cells do not express CD20, and thus many B-cell disorders are not treatable using anti-CD20 antibody therapy.

In addition to NHL there are several types of leukemias that result from disregulation of B cells. Chronic lymphocytic leukemia (also known as “chronic lymphoid leukemia” or “CLL”), is a type of adult leukemia caused by an abnormal accumulation of B lymphocytes. In CLL, the malignant lymphocytes may look normal and mature, but they are not able to cope effectively with infection. CLL is the most common form of leukemia in adults. Men are twice as likely to develop CLL as women. However, the key risk factor is age. Over 75% of new cases are diagnosed in patients over age 50. More than 10,000 cases are diagnosed every year and the mortality is almost 5,000 a year (American Cancer Society, 2006; and SEER Cancer Statistics Review). CLL is an incurable disease but progresses slowly in most cases. Many people with CLL lead normal and active lives for many years. Because of its slow onset, early-stage CLL is generally not treated since it is believed that early CLL intervention does not improve survival time or quality of life. Instead, the condition is monitored over time. Initial CLL treatments vary depending on the exact diagnosis and the progression of the disease. There are dozens of agents used for CLL therapy. Combination chemotherapy regimens such as FCR (fludarabine, cyclophosphamide and rituximab), and BR (Ibrutinib and rituximab) are effective in both newly-diagnosed and relapsed CLL. Allogeneic bone marrow (stem cell) transplantation is rarely used as a first-line treatment for CLL due to its risk.

Another type of leukemia is acute lymphoblastic leukemia (ALL), also known as acute lymphocytic leukemia. ALL is characterised by the overproduction and continuous multiplication of malignant and immature white blood cells (also known as lymphoblasts) in the bone marrow. ‘Acute’ refers to the undifferentiated, immature state of the circulating lymphocytes (“blasts”), and that the disease progresses rapidly with life expectancy of weeks to months if left untreated. ALL is most common in childhood with a peak incidence of 4-5 years of age. Children of age 12-16 die more easily from it than others. Currently, at least 80% of childhood ALL are considered curable. Under 4,000 cases are diagnosed every year and the mortality is almost 1,500 a year (American Cancer Society, 2006; and SEER Cancer Statistics Review).

The human CD19 molecule is a structurally distinct cell surface receptor expressed on the surface of human B cells, including, but not limited to, pre-B cells, B cells in early development {i.e., immature B cells), mature B cells through terminal differentiation into plasma cells, and malignant B cells. CD 19 is expressed by most pre-B acute lymphoblastic leukemias (ALL), non-Hodgkin's lymphomas, B cell chronic lymphocytic leukemias (CLL), pro-lymphocytic leukemias, hairy cell leukemias, common acute lymphocytic leukemias, and some Null-acute lymphoblastic leukemias (Nadler et al, J. Immunol., 131:244-250 (1983), Loken et al, Blood, 70:1316-1324 (1987), Uckun et al, Blood, 71:13-29 (1988), Anderson et al, 1984. Blood, 63:1424-1433 (1984), Scheuermann, Leuk. Lymphoma, 18:385-397 (1995)). The expression of CD 19 on plasma cells further suggests it may be expressed on differentiated B cell tumors such as multiple myeloma, plasmacytomas, Waldenstrom's tumors (Grossbard et al., Br. J. Haematol, 102:509-15(1998); Treon et al, Semin. Oncol, 30:248-52(2003)).

Therefore, the CD 19 antigen is a target for immunotherapy in the treatment of non-Hodgkin's lymphoma (including each the subtypes described herein), chronic lymphocytic leukemia and/or acute lymphoblastic leukemia.

Certain CD19 therapies have been shown. T cells expressing an anti-CD19 chimeric antigen receptor (CAR) including both CD3-ζ and the 4-BB costimulatory domain were administered to patients with advanced CLL. Kalos et al., T cells with Chimeric Antigen Receptors Have Potent Antitumor Effects and Can Establish Memory in Patients with Advanced Leukemia, Science Translational Medicine, vol. 3, no. 95 (10 Aug. 2011), which is incorporated by reference in its entirety. Sadelain et al., The promise and potential pitfalls of chimeric antigen receptors, Current Opinion in Immunology, Elsevier, vol. 21, no. 2, 2 Apr. 2009, which is incorporated by reference in its entirety, also describes anti-CD19 chimeric antigen receptors (CARs). Neither Kalos et al. nor Sadelain et al., however, describe the antibody specific for CD19 in combination with a BCL-2 inhibitor as exemplified herein.

The use of a CD19 antibody in non-specific B cell lymphomas is discussed in WO2007076950 (US2007154473), which are both incorporated by reference in their entireties, along with the cursory mention of Ibrutinib within a long list of potential combination partners, but fails either to teach the antibody exemplified herein or suggest the synergistic effects of the combination in the treatment of non-Hodgkin's lymphoma, chronic lymphocytic leukemia and/or acute lymphoblastic leukemia as exemplified herein.

The use of a CD19 antibody in CLL, NHL and ALL is described in Scheuermann et al., CD19 Antigen in Leukemia and Lymphoma Diagnosis and Immunotherapy, Leukemia and Lymphoma, Vol. 18, 385-397 (1995), which is incorporated by reference in its entirety, but fails to suggest the combination exemplified herein.

Additional antibodies specific for CD19 are described in WO2005012493 (U.S. Pat. No. 7,109,304), WO2010053716 (U.S. Ser. No. 12/266,999) (Immunomedics); WO2007002223 (US U.S. Pat. No. 8,097,703) (Medarex); WO2008022152 (Ser. No. 12/377,251) and WO2008150494 (Xencor), WO2008031056 (U.S. Ser. No. 11/852,106) (Medimmune); WO 2007076950 (U.S. Ser. No. 11/648,505) (Merck Patent GmbH); WO 2009/052431 (U.S. Ser. No. 12/253,895) (Seattle Genetics); and WO2010095031 (Ser. No. 12/710,442) (Glenmark Pharmaceuticals), WO2012010562 and WO2012010561 (International Drug Development), WO2011147834 (Roche Glycart), and WO 2012/156455 (Sanofi), which are all incorporated by reference in their entireties.

Combinations of antibodies specific for CD19 and other agents are described in WO2010151341 (U.S. Ser. No. 13/377,514) (The Feinstein Institute); U.S. Pat. No. 5,686,072 (University of Texas), and WO2002022212 (PCT/US01/29026) (IDEC Pharmaceuticals), WO2013/024097 (Ser. No. 14/126,928) (MorphoSys AG) and WO2013/024095 (Ser. No. 14/127,217) (MorphoSys AG), which are all incorporated by reference in their entireties.

Certain BCL-2 inhibitors are commercially available. VENCLEXTA™ (venetoclax), also known as GDC-0199, ABT-199, and RG7601 is a BCL-2 inhibitor indicated for the treatment of patients with chronic lymphocytic leukemia (CLL) with 17p deletion, as detected by an FDA approved test, who have received at least one prior therapy. Venetoclax is described in U.S. Pat. Nos. 8,546,399 and 9,174,982, which are all incorporated by reference in their entireties.

Despite the recent progress in the discovery and development of anti-cancer agents, many forms of cancer involving CD19-expressing tumors still have a poor prognosis. Thus, there is a need for improved methods for treating such forms of cancer.

### SUMMARY

Neither alone nor in combination does the prior art suggest the synergistic effects of the combination of the exemplified antibody and venetoclax in the treatment of non-Hodgkin's lymphoma, chronic lymphocytic leukemia and/or acute lymphoblastic leukemia.

In one aspect, the present disclosure relates to a synergistic combination of an antibody specific for CD19 and a BCL-2 inhibitor. Such combinations are useful in the treatment of B cell malignancies, such as, non-Hodgkin's lymphoma, chronic lymphocytic leukemia and/or acute lymphoblastic leukemia.

In vitro models are considered indicative of how a certain compound or combination of compounds would behave in humans.

MEC-1 cells (DSMZ #ACC497) are a chronic B-cell leukemia cell line. MEC-1 cells in the present in vitro model are indicative of how the combination will work in the treatment of chronic lymphoid leukemia (CLL) in humans.

In addition, when compounds are combined in vitro, one expects that the combination has only additive effects. Surprisingly, the inventors found that the combination of a particular antibody specific for CD19 and venetoclax mediated a synergistic level of specific cell killing in vitro in comparison to the antibody and venetoclax alone. Specifically, the inventors found that the combination of MOR00208 and venetoclax mediated a synergistic level of specific cell killing in vitro in MEC-1 cells compared to the antibody and venetoclax alone.

In summary, the combination of the exemplified anti-CD19 antibody and venetoclax behaved synergistically in models relevant to CLL. As CLL is a B cell related disorders and CD19 is highly expressed on B-cells, the exemplified combination would have the same mechanism of action and should also behave synergistically in the treatment of other B cell related disorders, e.g. ALL and NHL.

Therefore, the combination of the exemplified antibody specific for CD19 and venetoclax should be effective in the treatment of humans in non-Hodgkin's lymphoma, chronic lymphocytic leukemia and/or acute lymphoblastic leukemia. The expected efficacy of the combination of the antibody specific to CD19 exemplified and venetoclax will be confirmed in clinical trials.

As the mechanism of action of venetoclax and other BCL-2 inhibitors are similar, as they work by inhibiting the anti-apoptotic B-cell lymphoma-2 (Bcl-2) protein, leading to programmed cell death of cells, it is believed that synergy should also be seen when treating humans having non-Hodgkin's lymphoma, chronic lymphocytic leukemia and/or acute lymphoblastic leukemia with a combination of the exemplified anti-CD19 antibody and a BCL-2 inhibitor other than venetoclax.

As the exemplified anti-CD19 antibody and other anti-CD19 antibodies bind CD19, it is believed that synergy should also be seen when treating humans having non-Hodgkin's lymphoma, chronic lymphocytic leukemia and/or acute lymphoblastic leukemia with a combination of any anti-CD19 antibody and a BCL-2 inhibitor, e.g., venetoclax.

An aspect of the present disclosure comprises a combination wherein the antibody specific for CD19 comprises an HCDR1 region of sequence SYVMH (SEQ ID NO: 1), an HCDR2 region of sequence NPYNDG (SEQ ID NO: 2), an HCDR3 region of sequence GTYYYGTRVFDY (SEQ ID NO: 3), an LCDR1 region of sequence RSSKSLQNVNGNTYLY (SEQ ID NO: 4), an LCDR2 region of sequence RMSNLNS (SEQ ID NO: 5), and an LCDR3 region of sequence MQHLEYPIT (SEQ ID NO: 6) and venetoclax. In embodiments, the combination is synergistic. In preferred aspects, the combination is used for the treatment of non-Hodgkin's lymphoma, chronic lymphocytic leukemia and/or acute lymphoblastic leukemia.

### DESCRIPTION OF DRAWINGS

FIGS. 1-3 shows the cytotoxicity of MOR00208 and venetoclax in combination in MEC-1 cell line. MEC-1 cells were pre-treated with venetoclax for 24 hours.

FIG. 4 shows the amino acid sequence of the variable domains of MOR00208.

FIG. 5 shows the amino acid sequence of the Fc regions of MOR00208.

FIGS. 6-8 show Chou-Talay Combination Index curves of MOR00208 and venetoclax in MEC-1 cell line.

FIG. 9 MOR00208 in combination with ABT-199 showed superior reduction of tumor growth compared to either mono therapy in a s.c. Toledo SCID mouse model. Group mean values±standard error of mean (SEM) are depicted. Asterics indicated statistical siginificance of the tumor doubling time of the combination treatment vs the MOR0028 single treatment or the respective ABT-199 single treatment: *p-value<0.05, **p-value<0.01 and ****p-value<0.0001. Treatment started upon randomization at a median tumor volume of 266 mm³ (123-406 mm³) 23 days after tumor injection. MOR00208 (11 mg/kg) was administered by intraperitoneal injection (i.p.) twice weekly. ABT-199 (20 or 40 mg/kg) was administered by per os (p.o.) daily.

FIG. 10 MOR00208 in combination with ABT showed superior median survival compared to either monotherapy in a s.c. Toledo SCID mouse model. The Kaplan-Meier curves depicte the survival from randomization until the humane endpoint (Tumor volume of 2000 mm³). Treatment started upon randomization at a median tumor volume of 266 mm³ (123-406 mm³) 23 days after tumor injection. MOR00208 (11 mg/kg) was administered by intraperitoneal injection (i.p.) twice weekly. ABT-199 (20 or 40 mg/kg) was administered by per os (p.o.) daily.

### DETAILED DESCRIPTION OF THE INVENTION

“Synergy”, “synergism” or “synergistic” mean more than the expected additive effect of a combination. In respect to the in vitro studies the “synergy”, “synergism” or “synergistic” effect of a combination is determined herein by the methods of Chou et al., Clarke et al. and/or Webb et al. See Ting-Chao Chou, Theoretical Basis, Experimental Design, and Computerized Simulation of Synergism and Antagonism in Drug Combination Studies, Pharmacol Rev 58:621-681 (2006), which is incorporated by reference in its entirety. See also Clarke et al., Issues in experimental design and endpoint analysis in the study of experimental cytotoxic agents in vivo in breast cancer and other models, Breast Cancer Research and Treatment 46:255-278 (1997), which is incorporated by reference in its entirety. See also Webb, J. L. (1963) Enzyme and Metabolic Inhibitors, Academic Press, New York, which is incorporated by reference in its entirety.

The term “antibody” means monoclonal antibodies, including any isotype, such as, IgG, IgM, IgA, IgD and IgE. An IgG antibody is comprised of two identical heavy chains and two identical light chains that are joined by disulfide bonds. Each heavy and light chain contains a constant region and a variable region. Each variable region contains three segments called “complementarity-determining regions” (“CDRs”) or “hypervariable regions”, which are primarily responsible for binding an epitope of an antigen. They are referred to as CDR1, CDR2, and CDR3, numbered sequentially from the N-terminus. The more highly conserved portions of the variable regions outside of the CDRs are called the “framework regions”. An “antibody fragment” means an Fv, scFv, dsFv, Fab, Fab′ F(ab′)2 fragment, or other fragment, which contains at least one variable heavy or variable light chain, each containing CDRs and framework regions.

Bcl-2 (B-cell lymphoma 2), encoded in humans by the BCL2 gene, is the founding member of the Bcl-2 family of regulator proteins that regulate cell death (apoptosis), by either inducing (pro-apoptotic) or inhibiting (anti-apoptotic) apoptosis. Bcl-2 is refenced by the NCBI Gene number 596. Bcl-2 is specifically considered an important anti-apoptotic protein and is thus classified as an oncogene. Bcl-2 derives its name from B-cell lymphoma 2, as it is the second member of a range of proteins initially described in chromosomal translocations involving chromosomes 14 and 18 in follicular lymphomas. Orthologs (such as Bcl2 in mice) have been identified in numerous mammals for which complete genome data are available.

A “BCL-2 inhibitor” is a class of drug that functions by inhibiting anti-apoptotic B-cell lymphoma-2 (Bcl-2) protein, leading to programmed cell death of cells. BCL-2 inhibitor include venetoclax. Venetoclax is marketed by Abbvie and Genentech (trade name VENCLEXTA™, also known as GDC-0199, ABT-199, and RG7601). Venetoclax is currently labelled for the treatment of patients with chronic lymphocytic leukemia (CLL) with 17p deletion, as detected by an FDA approved test, who have received at least one prior therapy. The formula of venetoclax is 4-(4-{[2-(4-Chlorophenyl)-4,4-dimethyl-1-cyclohexen-1-yl]methyl}-1-piperazinyl)-N-({3-nitro-4-[(tetrahydro-2H-pyran-4-ylmethyl)amino]phenyl}sulfonyl)-2-(1H-pyrrolo[2,3-b]pyridin-5-yloxy)benzamide and has the following structure:

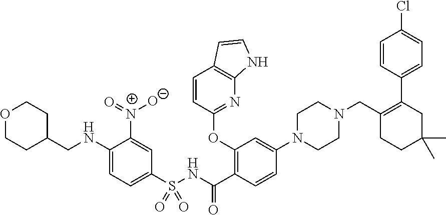 [SMILES: CC1(C)CCC(CN2CCN(c3ccc(C(=O)NS(=O)(=O)c4ccc(NCC5CCOCC5)c([N+](=O)[O-])c4)c(Oc4ccc5cc[nH]c5n4)c3)CC2)=C(c2ccc(Cl)cc2)C1]

“Venetoclax,” “ABT”, and “ABT-199” are used as synonyms herein.

### Other BCL-2 Inhibitors Include:

Genasense: An antisense oligonucleotide drug Genasense (G3139) was developed by Genta Incorporated to target Bcl-2. An antisense DNA or RNA strand is non-coding and complementary to the coding strand (which is the template for producing respectively RNA or protein). An antisense drug is a short sequence of RNA that hybridises with and inactivates mRNA, preventing the protein from being formed. Human lymphoma cell proliferation (with t(14;18) translocation) could be inhibited by antisense RNA targeted at the start codon region of Bcl-2 mRNA. In vitro studies led to the identification of Genasense, which is complementary to the first 6 codons of Bcl-2 mRNA. These showed successful results in Phase I/II trials for lymphoma. A large Phase III trial was launched in 2004. As of 2016, the drug had not been approved and its developer was out of business.

ABT-737 and ABT-263: In the mid-2000s, Abbott Laboratories developed a novel inhibitor of Bcl-2, Bcl-xL and Bcl-w, known as ABT-737. This compound is part of a group of BH3 mimetic small molecule inhibitors (SMI) that target these Bcl-2 family proteins, but not A1 or Mcl-1. ABT-737 is superior to previous BCL-2 inhibitors given its higher affinity for Bcl-2, Bcl-xL and Bcl-w. In vitro studies showed that primary cells from patients with B-cell malignancies are sensitive to ABT-737. In animal models, it improves survival, causes tumor regression and cures a high percentage of mice. In preclinical studies utilizing patient xenografts, ABT-737 showed efficacy for treating lymphoma and other blood cancers. Because of its unfavorable pharmacologic properties ABT-737 is not appropriate for clinical trials, while its derivative ABT-263 has similar activity on small cell lung cancer (SCLC) cell lines and has entered clinical trials.

“VH” refers to the variable region of an immunoglobulin heavy chain of an antibody, or antibody fragment. “VL” refers to the variable region of the immunoglobulin light chain of an antibody, or antibody fragment.

The term “CD19” refers to the protein known as CD19, having the following synonyms: B4, B-lymphocyte antigen CD19, B-lymphocyte surface antigen B4, CVID3, Differentiation antigen CD19, MGC12802, and T-cell surface antigen Leu-12.

Human CD19 has the amino acid sequence of:

(SEQ ID NO: 7)
MPPPRLLFFLLFLTPMEVRPEEPLVVKVEEGDNAVLQCLKGTSDGPTQQL
TWSRESPLKPFLKLSLGLPGLGIHMRPLAIWLFIFNVSQQMGGFYLCQPG
PPSEKAWQPGWTVNVEGSGELFRWNVSDLGGLGCGLKNRSSEGPSSPSGK
LMSPKLYVWAKDRPEIWEGEPPCLPPRDSLNQSLSQDLTMAPGSTLWLSC
GVPPDSVSRGPLSWTHVHPKGPKSLLSLELKDDRPARDMWVMETGLLLPR
ATAQDAGKYYCHRGNLTMSFHLEITARPVLWHWLLRTGGWKVSAVTLAYL
IFCLCSLVGILHLQRALVLRRKRKRMTDPIRRFFKVIPPPGSGPQNQYGN
VLSLPTPTSGLGRAQRWAAGLGGTAPSYGNPSSDVQADGALGSRSPPGVG
PEEEEGEGYEEPDSEEDSEFYENDSNLGQDQLSQDGSGYENPEDEPLGPE
DEDSFSNAESYENEDEELTQPVARTMDFLSPHGSAWDPSREATSLGSQSY
EDMRGILYAAPQLRSIRGQPGPNHEEDADSYENMDNPDGPDPAWGGGGRM
GTWSTR.

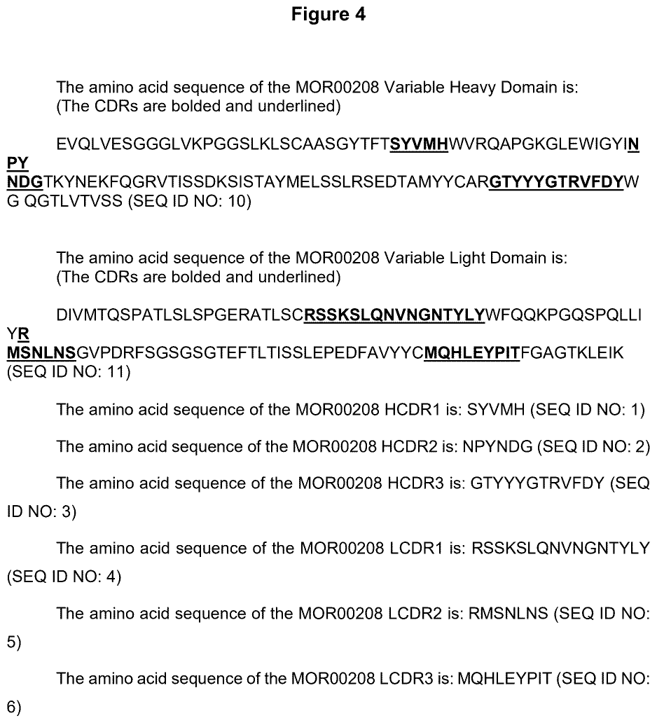

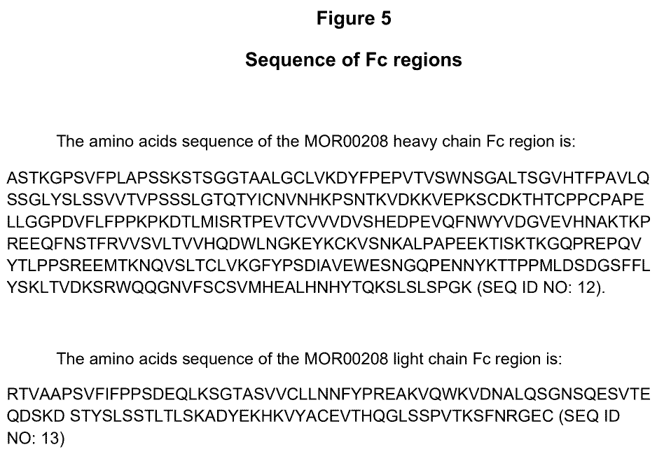

“MOR00208” is an anti-CD19 antibody. The amino acid sequence of the variable domains is provided in FIG. 4. The amino acid sequence of the heavy and light chain Fc regions of MOR00208 are provided in FIG. 5. “MOR00208” and “XmAb 5574” are used as synonyms to describe the antibody shown in FIGS. 4 and 5. The MOR00208 antibody is described in U.S. patent application Ser. No. 12/377,251, which is incorporated by reference in its entirety.

U.S. patent application Ser. No. 12/377,251 describes the antibody named 4G7 H1.52 Hybrid S239D/I332E/4G7 L1.155 (later named MOR00208) as follows:

>4G7 H1.52 Hybrid S239D/I332E
(SEQ ID NO: 14)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPDVFLFPPK
PKDTLMISRTPEVTCVVVDVSHEDPEVQFNWYVDGVEVHNAKTKPREEQF
NSTFRVVSVLTVVHQDWLNGKEYKCKVSNKALPAPEEKTISKTKGQPREP
QVYTLPPSREEMTKNQVSLTCLVKGFYPSDIAVEWESNGQPENNYKTIPP
MLDSDGSFFLYSKLTVDKSRWQQGNVFSCSVMHEALHNHYTQKSLSLSPG
K
>4G7 L1.155
(SEQ ID NO: 15)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

Additional antibodies specific for CD19 are described in U.S. Pat. No. 7,109,304 (Immunomedics), which is incorporated by reference in its entirety; U.S. application Ser. No. 11/917,750 (Medarex), which is incorporated by reference in its entirety; U.S. application Ser. No. 11/852,106 (Medimmune), which is incorporated by reference in its entirety; U.S. application Ser. No. 11/648,505 (Merck Patent GmbH), which is incorporated by reference in its entirety; U.S. Pat. No. 7,968,687 (Seattle Genetics), which is incorporated by reference in its entirety; and U.S. application Ser. No. 12/710,442 (Glenmark Pharmaceuticals), which is incorporated by reference in its entirety.

“Fc region” means the constant region of an antibody, which in humans may be of the IgG1, 2, 3, 4 subclass or others. The sequences of human Fc regions are available at IMGT, Human IGH C-REGIONS, www.imgt.org/IMGTrepertoire/Proteins/protein/human/IGH/IGHC/Hu_IGHCallgenes.html (retrieved on 16 May 2011).

“RefmAb33” is an antibody whose amino acid sequence is as follows:

Heavy chain including the Fc region:
(SEQ ID NO: 8)
QVTLRESGPALVKPTQTLTLTCTFSGFSLSTAGMSVGWIRQPPGKALEWL
ADIWWDDKKHYNPSLKDRLTISKDTSKNQVVLKVTNMDPADTATYYCARD
MIFNFYFDVWGQGTTVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPDVFLFPPKP
KDTLMISRTPEVICVVVDVSHEDPEVQFNWYVDGVEVHNAKTKPREEQFN
STFRVVSVLTVVHQDWLNGKEYKCKVSNKALPAPEEKTISKTKGQPREPQ
VYTLPPSREEMTKNQVSLTCLVKGFYPSDIAVEWESNGQPENNYKTTPPM
LDSDGSFFLYSKLTVDKSRWQQGNVFSCSVMHEALHNHYTQKSLSLSPG
K.
Light chain including the Fc region:
(SEQ ID NO: 9)
DIQMTQSPSTLSASVGDRVTITCSASSRVGYMHWYQQKPGKAPKLLIYDT
SKLASGVPSRFSGSGSGTEFTLTISSLQPDDFATYYCFQGSGYPFTFGGG
TKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC.

RefmAb33 is specific for RSV, and is used as isotype control, as it shares the same Fc region as MOR00208.

A “combination” means more than one item, e.g. a compound such as an antibody and venetoclax.

The present disclosure also relates to combinations, pharmaceuticals, and pharmaceutical compositions containing the described combinations. The two components of the combination of the present invention, e.g. the antibody specific for CD19 and venetoclax, may be administered together, simultaneously, separately or subsequently, either physically or in time.

Venetoclax is currently taken orally and is currently dosed once per day. MOR00208 is currently administered intravenously, and is currently dosed either once a week or once every two weeks.

Preferably, administration of both drugs allows for both drugs to be active in the patient at the same time. For example, if MOR208 is dosed weekly and venetoclax is dosed daily then the active substance of both drugs is present in the patient at the same time. In an embodiment, venetoclax, is administered prior to and/or separately from the administration of the antibody specific for CD19, e.g. MOR00208.

Simultaneously means that the two components are administered at a time where both components (drugs) are active in the patient at the same time. It is implied by “synergism” that both drugs are active in the patient at the same time.

Administered together can mean administered at the same time.

The components of the combination may be formulated in different pharmaceutical compositions. A pharmaceutical composition includes an active agent, eg. an antibody for therapeutic use in humans. A pharmaceutical composition may include acceptable carriers or excipients.

“Administered” or “administration” includes but is not limited to delivery by an injectable form, such as, for example, an intravenous, intramuscular, intradermal or subcutaneous route or mucosal route, for example, as a nasal spray or aerosol for inhalation or as an ingestable solution, capsule or tablet.

A “therapeutically effective amount” of a compound or combination refers to an amount sufficient to cure, alleviate or partially arrest the clinical manifestations of a given disease or disorder and its complications. The amount that is effective for a particular therapeutic purpose will depend on the severity of the disease or injury as well as on the weight and general state of the subject. It will be understood that determination of an appropriate dosage may be achieved, using routine experimentation, by constructing a matrix of values and testing different points in the matrix, all of which is within the ordinary skills of a trained physician or clinical scientist.

“Cross competes” means the ability of an antibody or other binding agent to interfere with the binding of other antibodies or binding agents to CD19 in a standard competitive binding assay. The ability or extent to which an antibody or other binding agent is able to interfere with the binding of another antibody or binding molecule to CD19, and, therefore whether it can be said to cross-compete according to the invention, can be determined using standard competition binding assays. One suitable assay involves the use of the Biacore technology (e.g. by using the BIAcore 3000 instrument (Biacore, Uppsala, Sweden)), which can measure the extent of interactions using surface plasmon resonance technology. Another assay for measuring cross-competing uses an ELISA-based approach. A high throughput process for “epitope binning” antibodies based upon their cross-competition is described in International Patent Application No. WO 2003/48731

The term “epitope” includes any protein determinant capable of specific binding to an antibody or otherwise interacting with a molecule. Epitopic determinants generally consist of chemically active surface groupings of molecules such as amino acids or carbohydrate or sugar side chains and can have specific three-dimensional structural characteristics, as well as specific charge characteristics. An epitope may be “linear” or “conformational.” The term “linear epitope” refers to an epitope with all of the points of interaction between the protein and the interacting molecule (such as an antibody) occur linearally along the primary amino acid sequence of the protein (continuous). The term “conformational epitope” refers to an epitope in which discontinuous amino acids that come together in three dimensional conformation. In a conformational epitope, the points of interaction occur across amino acid residues on the protein that are separated from one another.

“Binds the same epitope as” means the ability of an antibody or other binding agent to bind to CD19 and having the same epitope as the exemplified antibody. The epitopes of the exemplified antibody and other antibodies to CD19 can be determined using standard epitope mapping techniques. Epitope mapping techniques, well known in the art, include Epitope Mapping Protocols in Methods in Molecular Biology, Vol. 66 (Glenn E. Morris, Ed., 1996) Humana Press, Totowa, New Jersey. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al, (1984) Proc. Natl. Acad. Sci. USA 8:3998-4002; Geysen et al, (1985) Proc. Natl. Acad. Sci. USA 82:78-182; Geysen et al, (1986) Mol. Immunol. 23:709-715. Similarly, conformational epitopes are readily identified by determining spatial conformation of amino acids such as by, e.g., hydrogen/deuterium exchange, x-ray crystallography and two-dimensional nuclear magnetic resonance. See, e.g., Epitope Mapping Protocols, supra. Antigenic regions of proteins can also be identified using standard antigenicity and hydropathy plots, such as those calculated using, e.g., the Omiga version 1.0 software program available from the Oxford Molecular Group. This computer program employs the Hopp/Woods method, Hopp et al, (1981) Proc. Natl. Acad. Sci USA 78:3824-3828; for determining antigenicity profiles, and the Kyte-Doolittle technique, Kyte et al, (1982) J. Mol. Biol. 157:105-132; for hydropathy plots.

### Embodiments

An aspect of the present disclosure is a combination comprising an antibody specific for CD19 and a BCL-2 inhibitor for use in the treatment of non-Hodgkin's lymphoma, chronic lymphocytic leukemia and/or acute lymphoblastic leukemia. In embodiments, the combination is synergistic.

Herein, the combination of the exemplified anti-CD19 antibody and venetoclax behaved synergistically in in vitro models relevant to CLL. As CLL is a B cell related disorder and CD19 is highly expressed on B-cells, the exemplified combination should have the same mechanism of action and should also behave synergistically in the treatment of other B cell related disorders, e.g. ALL and NHL. Therefore, the combination of the exemplified antibody specific for CD19 and venetoclax should be effective in the treatment of humans in non-Hodgkin's lymphoma, chronic lymphocytic leukemia and/or acute lymphoblastic leukemia. The expected efficacy of the combination of the antibody specific to CD19 exemplified and venetoclax will be confirmed in clinical trials.

MEC-1 cells (DSMZ #ACC497), a chronic B-cell leukemia cell line, were tested. MEC-1 cells in the present in vitro model are indicative of how the combination will work in the treatment of chronic lymphoid leukemia (CLL) in humans. The Chou index values indicate clear synergism of the combination of MOR00208 and venetoclax in the specific killing of MEC-1 cells as compared to MOR00208 and venetoclax alone.

In summary, the combination of the exemplified anti-CD19 antibody and venetoclax behaved synergistically in models relevant to CLL.

Therefore, the combination of the exemplified antibody specific for CD19 and venetoclax should be effective in the treatment of humans in non-Hodgkin's lymphoma, chronic lymphocytic leukemia and/or acute lymphoblastic leukemia.

As the mechanism of action of venetoclax and other BCL-2 inhibitors are similar, it is believed that synergy should also be seen when treating humans having non-Hodgkin's lymphoma, chronic lymphocytic leukemia and/or acute lymphoblastic leukemia with a combination of the exemplified anti-CD19 antibody and a BCL-2 inhibitor other than venetoclax.

As the exemplified anti-CD19 antibody and other anti-CD19 antibodies bind CD19, it is believed that synergy should also be seen when treating humans having non-Hodgkin's lymphoma, chronic lymphocytic leukemia and/or acute lymphoblastic leukemia with a combination of any anti-CD19 antibody and a B-cell lymphoma-2 (Bcl-2) protein inhibitor, where the anti-CD19 antibody is, for example, described in U.S. patent application Ser. No. 12/377,251 (Xencor), WO2005012493, WO2010053716 (Immunomedics); WO2007002223 (Medarex); WO2008022152 (Xencor); WO2008031056 (Medimmune); WO 2007/076950 (Merck Patent GmbH); WO 2009/052431 (Seattle Genetics); and WO2010095031 (Glenmark Pharmaceuticals), all of which are incorporated by reference in their entireties.

In embodiments, the antibody specific for CD19 comprises an antibody that cross-competes with the antibody comprising an HCDR1 region of sequence SYVMH (SEQ ID NO: 1), an HCDR2 region of sequence NPYNDG (SEQ ID NO: 2), an HCDR3 region of sequence GTYYYGTRVFDY (SEQ ID NO: 3), an LCDR1 region of sequence RSSKSLQNVNGNTYLY (SEQ ID NO: 4), an LCDR2 region of sequence RMSNLNS (SEQ ID NO: 5), and an LCDR3 region of sequence MQHLEYPIT (SEQ ID NO: 6).

In embodiments, the antibody specific for CD19 comprises an antibody that binds to the same epitope as an antibody comprising an HCDR1 region of sequence SYVMH (SEQ ID NO: 1), an HCDR2 region of sequence NPYNDG (SEQ ID NO: 2), an HCDR3 region of sequence GTYYYGTRVFDY (SEQ ID NO: 3), an LCDR1 region of sequence RSSKSLQNVNGNTYLY (SEQ ID NO: 4), an LCDR2 region of sequence RMSNLNS (SEQ ID NO: 5), and an LCDR3 region of sequence MQHLEYPIT (SEQ ID NO: 6).

In embodiments, the antibody specific for CD19 comprises an HCDR1 region of sequence SYVMH (SEQ ID NO: 1), an HCDR2 region of sequence NPYNDG (SEQ ID NO: 2), an HCDR3 region of sequence GTYYYGTRVFDY (SEQ ID NO: 3), an LCDR1 region of sequence RSSKSLQNVNGNTYLY (SEQ ID NO: 4), an LCDR2 region of sequence RMSNLNS (SEQ ID NO: 5), and an LCDR3 region of sequence MQHLEYPIT (SEQ ID NO: 6).

In embodiments, the antibody specific for CD19 has a cytotoxic activity. In embodiments, the antibody specific for CD19 comprises a constant region having ADCC inducing activity. In embodiments, the antibody specific for CD19 induces ADCC.

In embodiments, the antibody specific for CD19 induces ADCC and comprises an HCDR1 region of sequence SYVMH (SEQ ID NO: 1), an HCDR2 region of sequence NPYNDG (SEQ ID NO: 2), an HCDR3 region of sequence GTYYYGTRVFDY (SEQ ID NO: 3), an LCDR1 region of sequence RSSKSLQNVNGNTYLY (SEQ ID NO: 4), an LCDR2 region of sequence RMSNLNS (SEQ ID NO: 5), and an LCDR3 region of sequence MQHLEYPIT (SEQ ID NO: 6).

In embodiments, the antibody specific for CD19 comprises an HCDR1 region of sequence SYVMH (SEQ ID NO: 1), an HCDR2 region of sequence NPYNDG (SEQ ID NO: 2), an HCDR3 region of sequence GTYYYGTRVFDY (SEQ ID NO: 3), an LCDR1 region of sequence RSSKSLQNVNGNTYLY (SEQ ID NO: 4), an LCDR2 region of sequence RMSNLNS (SEQ ID NO: 5), and an LCDR3 region of sequence MQHLEYPIT (SEQ ID NO: 6) and a constant region having ADCC inducing activity.

In embodiments, the antibody specific for CD19 comprises a variable heavy chain of the sequence EVOLVESGGGLVKPGGSLKLSCAASGYTFTSYVMHWVRQAPGKGLEWIGYINPY NDGTKYNEKFQGRVTISSDKSISTAYMELSSLRSEDTAMYYCARGTYYYGTRVFDYWG QGTLVTVSS (SEQ ID NO: 10) and a variable light chain of the sequence DIVMTQSPATLSLSPGERATLSCRSSKSLQNVNGNTYLYWFQQKPGQSPQLLIYR MSNLNSGVPDRFSGSGSGTEFTLTISSLEPEDFAVYYCMQHLEYPITFGAGTKLEIK (SEQ ID NO: 11).

In certain embodiments, said antibody comprises a heavy chain constant domain of the sequence

(SEQ ID NO: 12)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPDVFLFPPKPKDTLMISRTPEVTCVVVDVS
HEDPEVQFNWYVDGVEVHNAKTKPREEQFNSTFRVVSVLTVVHQDWLNGK
EYKCKVSNKALPAPEEKTISKTKGQPREPQVYTLPPSREEMTKNQVSLTC
LVKGFYPSDIAVEWESNGQPENNYKTTPPMLDSDGSFFLYSKLTVDKSRW
QQGNVFSCSVMHEALHNHYTQKSLSLSPGK.

In embodiments, the antibody specific for CD19 comprises a light chain constant domain of the sequence

(SEQ ID NO: 13)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.

In embodiments, the BCL-2 inhibitor is venetoclax.

In embodiments, the components of the combination, the antibody specific for CD19 and venetoclax, are administered separately. In an embodiment, venetoclax is administered prior to administration of the antibody specific for CD19.

In embodiments, the components of the combination are administered at a time where both components (drugs) are active in the patient at the same time. It is implied by “synergism” that both drugs are active in the patient at the same time. In embodiments, the components of the combination are administered together, simultaneously, separately or subsequently, either physically or in time. In embodiments, the components of the combination are administered simultaneously.

In embodiments, the combination is a pharmaceutical composition. In embodiments, the composition comprises an acceptable carrier. In embodiments, the combination is administered in an effective amount.

An aspect of the present disclosure comprises an antibody specific for CD19 comprising an HCDR1 region of sequence SYVMH (SEQ ID NO: 1), an HCDR2 region of sequence NPYNDG (SEQ ID NO: 2), an HCDR3 region of sequence GTYYYGTRVFDY (SEQ ID NO: 3), an LCDR1 region of sequence RSSKSLQNVNGNTYLY (SEQ ID NO: 4), an LCDR2 region of sequence RMSNLNS (SEQ ID NO: 5), and an LCDR3 region of sequence MQHLEYPIT (SEQ ID NO: 6) for the treatment of non-Hodgkin's lymphoma, chronic lymphocytic leukemia and/or acute lymphoblastic leukemia, wherein said antibody is used in a synergistic combination with venetoclax.

An aspect of the present disclosure comprises an antibody specific for CD19 comprising an HCDR1 region of sequence SYVMH (SEQ ID NO: 1), an HCDR2 region of sequence NPYNDG (SEQ ID NO: 2), an HCDR3 region of sequence GTYYYGTRVFDY (SEQ ID NO: 3), an LCDR1 region of sequence RSSKSLQNVNGNTYLY (SEQ ID NO: 4), an LCDR2 region of sequence RMSNLNS (SEQ ID NO: 5), and an LCDR3 region of sequence MQHLEYPIT (SEQ ID NO: 6) for the treatment of non-Hodgkin's lymphoma, chronic lymphocytic leukemia and/or acute lymphoblastic leukemia, wherein said antibody is used in a combination with venetoclax. In an embodiment said antibody is administered in combination with venetoclax. In another embodiment said antibody is used in a combination with venetoclax, wherein said antibody and venetoclax are administered separately. In further embodiments, said antibody is administered prior to venetoxlax. In further embodiments venetoclax is administered prior to said antibody.

An aspect of the present disclosure comprises a synergistic combination of an antibody specific for CD19 comprising an HCDR1 region of sequence SYVMH (SEQ ID NO: 1), an HCDR2 region of sequence NPYNDG (SEQ ID NO: 2), an HCDR3 region of sequence GTYYYGTRVFDY (SEQ ID NO: 3), an LCDR1 region of sequence RSSKSLQNVNGNTYLY (SEQ ID NO: 4), an LCDR2 region of sequence RMSNLNS (SEQ ID NO: 5), and an LCDR3 region of sequence MQHLEYPIT (SEQ ID NO: 6) and venetoclax for the treatment of non-Hodgkin's lymphoma, chronic lymphocytic leukemia and/or acute lymphoblastic leukemia. In embodiments, the non-Hodgkin's lymphoma is selected from the group consisting of follicular lymphoma, small lymphocytic lymphoma, mucosa-associated lymphoid tissue, marginal zone, diffuse large B cell, Burkitt's, and mantle cell.

In embodiments, the non-Hodgkin's lymphoma is follicular lymphoma. In embodiments, the non-Hodgkin's lymphoma is small lymphocytic lymphoma. In embodiments, the non-Hodgkin's lymphoma is mucosa-associated lymphoid tissue. In embodiments, the non-Hodgkin's lymphoma is marginal zone lymphoma. In embodiments, the non-Hodgkin's lymphoma is diffuse large B cell lymphoma. In embodiments, the non-Hodgkin's lymphoma is Burkitt's lymphoma. In embodiments, the non-Hodgkin's lymphoma is mantle cell lymphoma.

In embodiments, the combination is for the treatment of chronic lymphocytic leukemia. In embodiments, the combination is for the treatment of acute lymphoblastic leukemia.

Another aspect comprises a method of treating non-Hodgkin's lymphoma, chronic lymphocytic leukemia and/or acute lymphoblastic leukemia in an individual in need thereof, which method comprises administration of an antibody specific for CD19 and a BCL-2 inhibitor. In embodiments of the method, the antibody specific for CD19 comprises an HCDR1 region of sequence SYVMH (SEQ ID NO: 1), an HCDR2 region of sequence NPYNDG (SEQ ID NO: 2), an HCDR3 region of sequence GTYYYGTRVFDY (SEQ ID NO: 3), an LCDR1 region of sequence RSSKSLQNVNGNTYLY (SEQ ID NO: 4), an LCDR2 region of sequence RMSNLNS (SEQ ID NO: 5), and an LCDR3 region of sequence MQHLEYPIT (SEQ ID NO: 6). In embodiments of the method, the antibody comprises the exemplified antibody specific for CD19. In embodiments of the method the BCL-2 inhibitor is venetoclax.

Another aspect includes a use of an antibody specific for CD19 wherein said antibody comprises an HCDR1 region of sequence SYVMH (SEQ ID NO: 1), an HCDR2 region of sequence NPYNDG (SEQ ID NO: 2), an HCDR3 region of sequence GTYYYGTRVFDY (SEQ ID NO: 3), an LCDR1 region of sequence RSSKSLQNVNGNTYLY (SEQ ID NO: 4), an LCDR2 region of sequence RMSNLNS (SEQ ID NO: 5), and an LCDR3 region of sequence MQHLEYPIT (SEQ ID NO: 6) in the manufacture of a medicament for the treatment of non-Hodgkin's lymphoma, chronic lymphocytic leukemia and/or acute lymphoblastic leukemia in synergistic combination with venetoclax.

### Examples

### Example 1: Cytotoxicity of MEC-1 Cells Using MOR00208 and Venetoclax Alone and in Combination

### Materials

Cell lines tested: MEC-1 cells (DSMZ #ACC497). Culture conditions of cell lines used are according to supplier's information. Cell Medium: Iscove's Modified Dulbecco's Medium (IMDM), Invitrogen, Cat No.: 31980; RPMI1640, Invitrogen, Cat No.: 31870; GlutaMAX, Invitrogen, CAT No.: 35050; FCS: Sigma CAT No.: F7524 LOT No.: 111M3396. NKs: RPMI1640, with GlutaMAX, Invitrogen, Cat No.: 31870, 10% FCS; Biocoll: Biochrome AG CAT No.: L6115 LOT No.: 0034D; MACS NK cell isolation kit: Miltenyi Biotec CAT No.: 130-092-657 LOT No.: 5150402327; venetoclax: Selleck Chem. CAT No.: S8048 LOT No.: S804803; FCS: Sigma CAT No.: F7524 LOT No.: 111M3396; and RefmAb33 (anti-RSV) with same Fc region as MOR00208.

### Methods

The cytotoxic potential of MOR00208 and venetoclax alone and in combination were tested in the MEC-1 cell line (CLL). Target cell killing is measured using the following parameters: venetoclax mono treatment at concentrations of 3 μM, 6.5 μM, and 10 μM; MOR00208 mono treatment at concentrations of 0.01 μM, 0.1 μM, 1 μM, 10 μM, 100 μM and 10 nM, and the combination treatment of the listed venetoclax and MOR00208 concentrations. The following are used as controls: RefmAb33, NK cells alone, MEC-1 cells alone or DMSO. In the venetoclax mono groups as well as in the MOR00208+venetoclax combination groups, target cells were pretreated with venetoclax or with DMSO control for 24 hours followed by removal of the dead cells prior to the ADCC assay. A dead cell removal kit was used to remove cells killed by the cytotoxic effect of venetoclax. The dead cell removal kit was implemented to mimick the removal of dead cells that also occurs in vivo and to prevent false negative interference of the dead cells with the subsequent ADCC assay. For the ADCC assay, target cells are counted and stained using CFSE at a final concentration of 1 μg/mL. For the control group, i.e. DMSO treated target cells, the effector: target (E:T) cell ratio was adjusted to 2:1, i.e. 1×10E6/mL effector cells (NK cells) and 5×10E5/mL target cells (MEC-1 cells). For the venetoclax mono group as well as for the MOR00208+venetoclax combination group the number of target cells was reduced according to the observed cytotoxic effects of venetoclax during the 24 hour treatment while the number of effector cells was kept constant at 1×10⁶/mL. The ADCC assays were performed as follows: using 96 well plates, 100 μL of MEC-1 target cell suspension was added per well, followed by 100 μL NK effector cell suspension per well. Mixed cell suspensions were centrifuged and resuspended in 100 μL antibody containing medium or the according control solution. The antibodies were diluted in a range of 10 nM-0.01 pM (corresponds to 1.5 μg/mL-1.5 μg/mL) in medium. The ADCC assays were incubated for 2 hours in a CO2-incubator at 37° C. After 10 minutes incubation on ice, 50 μL DAPI solution was added to each well (final concentration 1 μg/mL) and incubated for additional 10 minutes on ice. The ADCC measurements were performed on a BD FACSVerse instrument. Dead target cells were identified as DAPI positive cells.

### Data

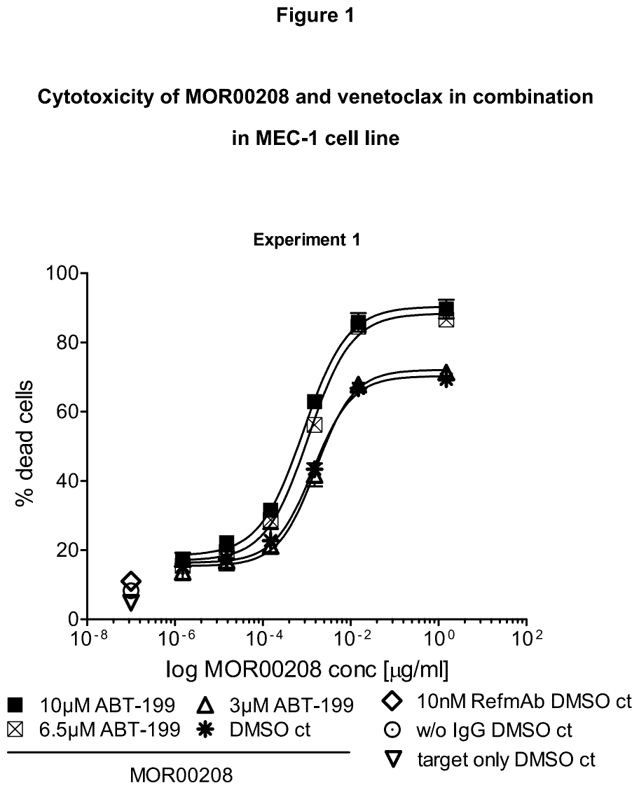

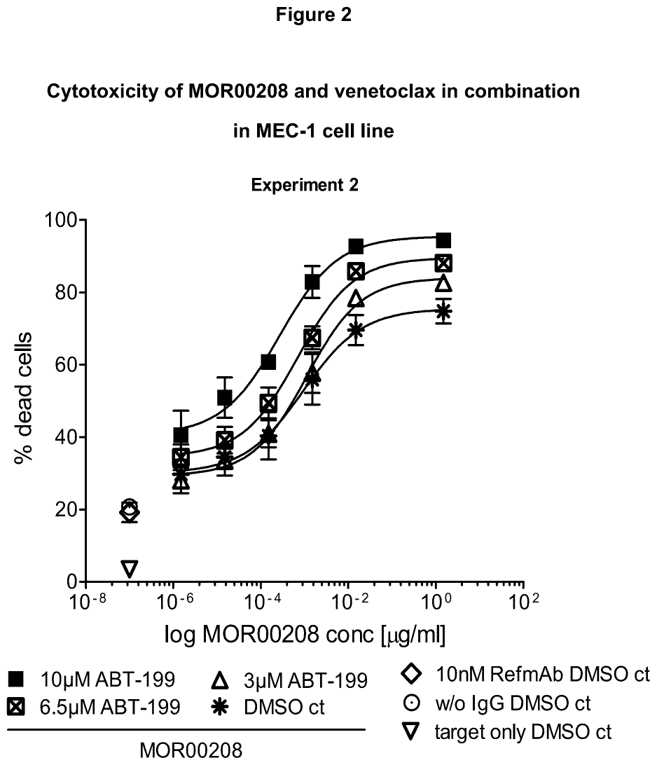

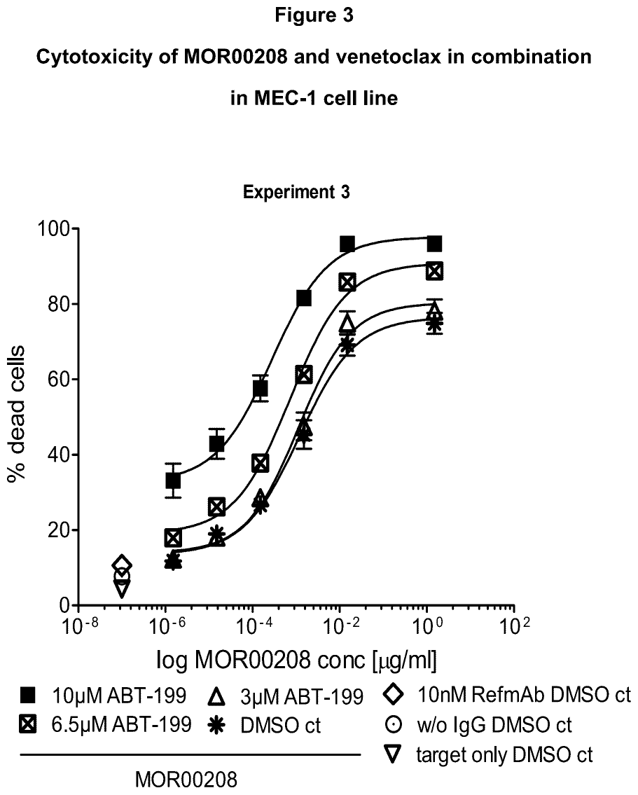

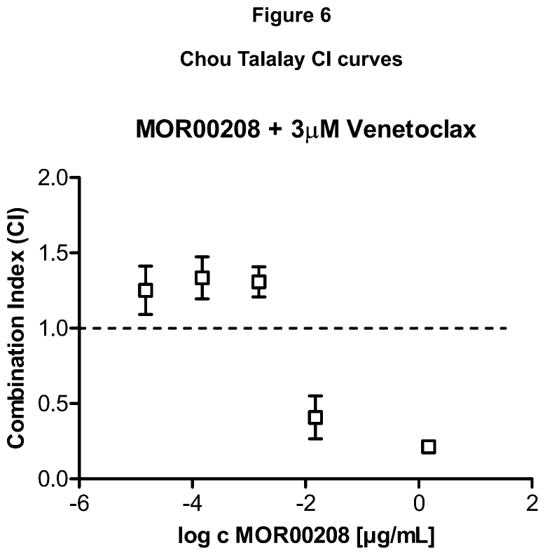

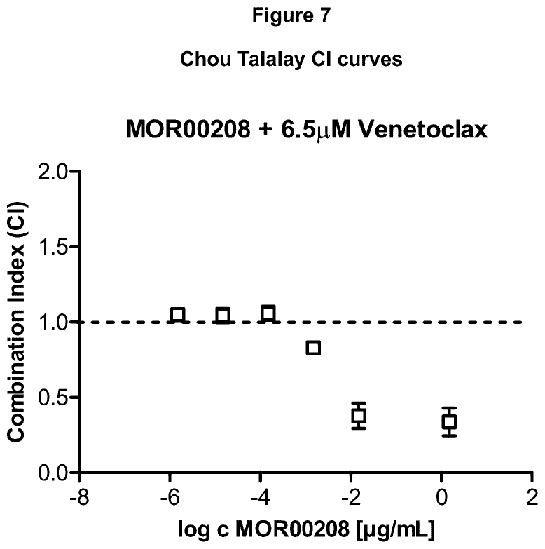

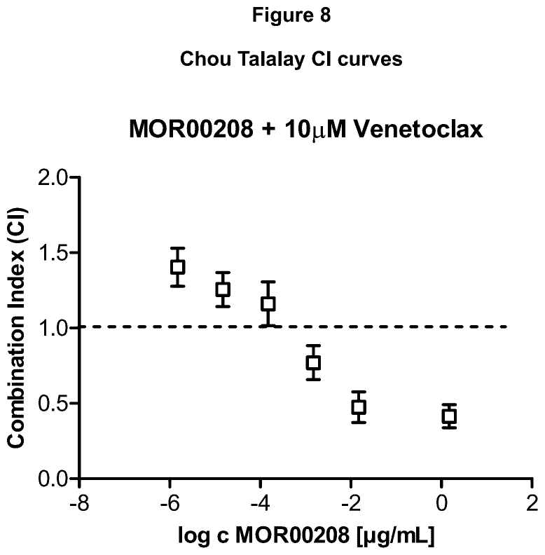

In total, three independent experiments were performed in order to determine the cytotoxic potential of the combination of MOR00208 and venetoclax. Individual raw data tables for all three experiments are shown in Tables 1-6. Individual ADCC dose response curves for all three experiments are shown in FIGS. 1-3. Mean (+/−SEM) combination index curves of all three experiments per each venetoclax concentration are shown in FIGS. 6-8.

### Experiment 1:

TABLE 1
Mono activity of MOR00208 and venetoclax

| Actual MOR00208 | Actual MOR00208 |
|---|---|
| Dose [μg/mL] | Effect [% dead cells] |
| 1.50E−06 | 15.3 |
| 1.50E−05 | 17.4 |
| 1.50E−04 | 22.8 |
| 1.50E−03 | 43.4 |
| 1.50E−02 | 66.8 |
| 1.50E+00 | 69.4 |
| Actual Venetoclax | Actual Venetoclax |
| Dose [μM] | Effect [% dead cells] |
| 3.0 | 8.9 |
| 6.5 | 17.7 |
| 10.0 | 38.8 |

TABLE 2
Combination activity of MOR00208 and venetoclax

| Actual | Actual | Actual MOR00208 + | Theoretical | Theoretical | |
|---|---|---|---|---|---|
| MOR00208 | Venetoclax | Venetoclax Effect | MOR00208 | Venetoclax | Combination |
| Dose [μg/mL] | Dose [μM] | [% dead cells] | Dose [μg/mL] | Dose [μM] | Index |
| 1.50E−05 | 3.0 | 16.9 | 1.41E−05 | 6.3 | 1.54 |
| 1.50E−04 | | 21.2 | 1.32E−04 | 7.2 | 1.55 |
| 1.50E−03 | | 41.7 | 1.25E−03 | 10.4 | 1.49 |
| 1.50E−02 | | 67.8 | 3.04E−02 | 15.1 | 0.69 |
| 1.50E+00 | | 71.3 | * 1.00E+03 | 16.0 | 0.19 |
| 1.50E−05 | 6.5 | 19.5 | 8.47E−05 | 6.9 | 1.12 |
| 1.50E−04 | | 28.7 | 4.10E−04 | 8.5 | 1.13 |
| 1.50E−03 | | 56.2 | 4.04E−03 | 12.7 | 0.88 |
| 1.50E−02 | | 84.5 | * 1.00E+03 | 21.9 | 0.30 |
| 1.50E+00 | | 86.6 | * 1.00E+03 | 23.9 | 0.27 |
| 1.50E−06 | 10.0 | 17.3 | 2.33E−05 | 6.4 | 1.63 |
| 1.50E−05 | | 22.1 | 1.63E−04 | 7.4 | 1.45 |
| 1.50E−04 | | 31.6 | 5.43E−04 | 8.9 | 1.40 |
| 1.50E−03 | | 62.9 | 9.14E−03 | 14.0 | 0.88 |
| 1.50E−02 | | 85.8 | * 1.00E+03 | 23.0 | 0.43 |
| 1.50E+00 | | 89.6 | * 1.00E+03 | 28.4 | 0.35 |

* Infinite theoretical MOR00208 dose as actual MOR00208 effect saturates below MOR00208 + Venetoclax effect

### Experiment 2:

TABLE 3
Mono activity of MOR00208 and venetoclax

| Actual MOR00208 | Actual MOR00208 |
|---|---|
| Dose [μg/mL] | Effect [% dead cells] |
| 1.50E−06 | 29.7 |
| 1.50E−05 | 34.5 |
| 1.50E−04 | 40.3 |
| 1.50E−03 | 55.9 |
| 1.50E−02 | 69.6 |
| 1.50E+00 | 74.8 |
| Actual Venetoclax | Actual Venetoclax |
| Dose [μM] | Effect [% dead cells] |
| 3.0 | 19.9 |
| 6.5 | 32.4 |
| 10.0 | 73.9 |

TABLE 4
Combination activity of MOR00208 and venetoclax

| Actual | Actual | Actual MOR00208 + | Theoretical | Theoretical | |
|---|---|---|---|---|---|
| MOR00208 | Venetoclax | Venetoclax Effect | MOR00208 | Venetoclax | Combination |
| Dose [μg/mL] | Dose [μM] | [% dead cells] | Dose [μg/mL] | Dose [μM] | Index |
| 1.50E−05 | 3.0 | 33.5 | 1.94E−05 | 6.6 | 1.23 |
| 1.50E−04 | | 40.9 | 1.55E−04 | 7.3 | 1.38 |
| 1.50E−03 | | 57.8 | 1.88E−03 | 8.6 | 1.15 |
| 1.50E−02 | | 78.4 | * 1.00E+03 | 10.6 | 0.28 |
| 1.50E+00 | | 82.6 | * 1.00E+03 | 11.2 | 0.27 |
| 1.50E−06 | 6.5 | 34.4 | 2.90E−05 | 6.7 | 1.02 |
| 1.50E−05 | | 39.1 | 1.09E−04 | 7.1 | 1.05 |
| 1.50E−04 | | 49.4 | 5.91E−04 | 7.9 | 1.07 |
| 1.50E−03 | | 67.4 | 1.00E−02 | 9.4 | 0.84 |
| 1.50E−02 | | 85.8 | * 1.00E+03 | 11.9 | 0.55 |
| 1.50E+00 | | 88.1 | * 1.00E+03 | 12.5 | 0.52 |
| 1.50E−06 | 10.0 | 40.5 | 1.45E−04 | 7.2 | 1.39 |
| 1.50E−05 | | 50.9 | 7.25E−04 | 8.0 | 1.26 |
| 1.50E−04 | | 60.7 | 2.89E−03 | 8.8 | 1.19 |
| 1.50E−03 | | 82.9 | * 1.00E+03 | 11.3 | 0.89 |
| 1.50E−02 | | 92.7 | * 1.00E+03 | 15.0 | 0.67 |
| 1.50E+00 | | 94.4 | * 1.00E+03 | 17.6 | 0.57 |

* Infinite theoretical MOR00208 dose as actual MOR00208 effect saturates below MOR00208 + Venetoclax effect

### Experiment 3:

TABLE 5
Mono activity of MOR00208 and venetoclax

| Actual MOR00208 | Actual MOR00208 |
|---|---|
| Dose [μg/mL] | Effect [% dead cells] |
| 1.50E−06 | 11.8 |
| 1.50E−05 | 18.9 |
| 1.50E−04 | 26.6 |
| 1.50E−03 | 45.3 |
| 1.50E−02 | 69.1 |
| 1.50E+00 | 74.8 |
| Actual Venetoclax | Actual Venetoclax |
| Dose [μM] | Effect [% dead cells] |
| 3.0 | 7.7 |
| 6.5 | 16.3 |
| 10.0 | 48.1 |

TABLE 6
Combination activity of MOR00208 and venetoclax

| Actual | Actual | Actual MOR00208 + | Theoretical | Theoretical | |
|---|---|---|---|---|---|
| MOR00208 | Venetoclax | Venetoclax Effect | MOR00208 | Venetoclax | Combination |
| Dose [μg/mL] | Dose [μM] | [% dead cells] | Dose [μg/mL] | Dose [μM] | Index |
| 1.50E−05 | 3.0 | 18.1 | 2.90E−05 | 6.4 | 0.99 |
| 1.50E−04 | | 28.6 | 2.18E−04 | 7.9 | 1.07 |
| 1.50E−03 | | 47.5 | 1.52E−03 | 10.2 | 1.28 |
| 1.50E−02 | | 74.9 | 2.79E−01 | 15.4 | 0.25 |
| 1.50E+00 | | 78.1 | * 1.00E+03 | 16.6 | 0.18 |
| 1.50E−06 | 6.5 | 17.9 | 2.67E−05 | 6.4 | 1.08 |
| 1.50E−05 | | 26.2 | 1.57E−04 | 7.5 | 0.96 |
| 1.50E−04 | | 37.8 | 6.02E−04 | 9.0 | 0.97 |
| 1.50E−03 | | 61.3 | 6.52E−03 | 12.2 | 0.76 |
| 1.50E−02 | | 85.8 | * 1.00E+03 | 22.3 | 0.29 |
| 1.50E+00 | | 88.7 | * 1.00E+03 | 29.4 | 0.22 |
| 1.50E−06 | 10.0 | 33.1 | 3.71E−04 | 8.4 | 1.19 |
| 1.50E−05 | | 42.9 | 9.83E−04 | 9.6 | 1.06 |
| 1.50E−04 | | 57.6 | 4.20E−03 | 11.6 | 0.90 |
| 1.50E−03 | | 81.6 | * 1.00E+03 | 18.4 | 0.54 |
| 1.50E−02 | | 95.9 | * 1.00E+03 | 31.0 | 0.32 |
| 1.50E+00 | | 96.0 | * 1.00E+03 | 31.0 | 0.32 |

* Infinite theoretical MOR00208 dose as actual MOR00208 effect saturates below MOR00208 + Venetoclax effect

### Calculation of Synergism

Combination Index (CI) calculations are completed in order to determine synergy of the combination of the exemplified anti-CD19 antibody and venetoclax as compared to MOR00208 and venetoclax alone. Such calculations are described in Ting-Chao Chou, Theoretical Basis, Experimental Design, and Computerized Simulation of Synergism and Antagonism in Drug Combination Studies, Pharmacol Rev 58:621-681 (2006), which is incorporated by reference in its entirety and Chou T C, Talalay P, Quantitative analysis of dose-effect relationships: the combined effects of multiple drugs or enzyme inhibitors. Adv Enzyme Regul 22:27-55 (1984), which is incorporated by reference in its entirety. The methods of Chou-Talalay are carried out using the CI-isobol method.

### Median Effect Equation

The median-effect equation models the effect of an inhibitor (such as a drug) as Fₐ/Fᵤ=(D/D50)\hat{ }m, where D is the dose, Fₐ and Fᵤ is the fraction of the system affected and unaffected by the dose D (Fₐ+Fᵤ=1); D50 is the dose producing the median effect (e.g. IC50, ED50, LD50). The constant m determines the shape of the dose-effect curve. We used GraphPad Prism to carry out a nonlinear regression calculation to estimate the parameters m and D50.

### CI-Isobol Method

The CI-isobol method provides a quantitative assessment of synergism between drugs. A combination index (CI) is estimated from dose-effect data of single and combined drug treatments. A value of CI less than 1 indicates synergism; CI=1 indicates additive effect; and CI>1 indicates antagonism. Drug interaction (synergism or antagonism) is more pronounced the farther a CI value is from 1. Formally, the combination index (CI) of a combined drug treatment is defined as CI=D₁/Dₓ₁+D₂/Dₓ₂. Here D1 and D2 are the doses of drug 1 and drug 2 of the combination, respectively; and Dx1, and Dx2 is the dose of a treatment with only drug 1 and drug 2 that would give the same effect as that of the combination. The doses Dx1 and Dx2 need to be estimated from the dose-effect data of single drug treatments. Essentially, a median effect equation is fitted to the data of each drug. From the median effect equation of a drug, we can estimate the dose (i.e. D) necessary to produce an effect (i.e. Fa, Fu). The further a point lies from the additive line, the bigger the different between 1 and its CI, thus the stronger the (synergistic or antagonistic) effect is.

### Results

Mean (+/−SEM) combination index curves of all three experiments for each venetoclax concentration are shown in FIGS. 6-8. The combination index values indicate clear synergism of the combination of MOR00208 and venetoclax in the specific killing of MEC-1 cells as compared to MOR00208 and venetoclax alone. Very low doses of MOR00208 have little or no effect in the exemplified ADCC cell killing assays. Accoridngly, the CI values at very low doses of MOR00208 show a value at or slightly above 1, which value represents only the acivity of venetoclax. At MOR00208 concentrations, where MOR00208 alone shows normal cell killing activity, clear synergism is shown by the CI values less than 1, which represents the activity of MOR00208 and venetoclax. The highest concentration of MOR00208 exemplified herein is obtained in the ongoing clinical trials, where MOR00208 is dosed at 12 mg/kg once weekly. Accordingly it is believed that the exemplified in vitro model is predictive of activity in humans. Therefore, the combination of MOR00208 and venetoclax should also behave synergistically in the treatment of non-Hodgkin's lymphoma (NHL), chronic lymphoid leukemia (CLL), and acute lymphoblastic leukemia (ALL) in humans.

It is to be understood that the description, specific examples and data, while indicating exemplary embodiments, are given by way of illustration and are not intended to limit the present invention. Various changes and modifications within the present invention will become apparent to the skilled artisan from the discussion, disclosure and data contained herein, and thus are considered part of the invention.

Example 2: Efficacy of MOR00208 in Combination with ABT-199 in a Subcutaneous Human Lymphoma Cell Xenograft Model in SCID Mice

The efficacy benefit of MOR00208 in combination with venetoclax (ABT-199) was further studied in a SCID mouse model with subcutaneous TOLEDO human lymphoma cell tumors. Tumor growth and mortality were assessed.

### Materials & Methods

Toledo cell line and culture medium were purchased and provided by Oncodesign. Tumor cells were grown as monolayer at 37° C. in a humidified atmosphere (5% CO₂, 95% air). The culture medium was RPMI 1640 containing 2 mM L-glutamine (ref: BE12-702F, Lonza, Verviers, Belgium) supplemented with 10% fetal bovine serum (ref: P30-1506, Lonza), HBSS (ref: BE10-543F), Glucose (ref: G8769, Sigma, France), Hepes (ref: BE17-737E, Lonza) and Sodium pyruvate (ref: BE13-115E, Lonza). The cells are adherent to plastic flasks. For experimental use, tumor cells were detached from the culture flask by a 5-minute treatment with trypsin-versene (ref: BE02-007E, Lonza), in Hanks' medium without calcium or magnesium (ref: BE10-543F, Lonza) and neutralized by addition of complete culture medium. The cells were counted in a hemocytometer and their viability was assessed by 0.25% trypan blue exclusion assay.

Tumors were induced by subcutaneous injection of 10×10⁶ of Toledo cells in 200 μl of RPMI 1640 containing matrigel (50:50, v:v, ref: 356237, BD Biosciences, France) into the right flank of SCID mice. At D23, when tumors reached a mean volume of 256±68 mm³, 60 mice were randomized according to their individual tumor volume into 6 groups each of 10 animals using Vivo Manager® software (Biosystemes, Couternon, France). A statistical test (analysis of variance, ANOVA) was performed to test for homogeneity between groups. MOR208 was injected intraperitoneally (IP) into the peritoneal cavity of mice. Combined substance was administered by oral gavage (per os, PO) via a gavage tube.

The treatment schedule initiated at D23 was as follows:

Animals from group 1 received one daily PO administration of PEP and one IP injection twice a week of PBS.

Animals from group 2 received daily PO administration of ABT-199 at 20 mg/kg/adm

Animals from group 3 received daily PO administration of ABT-199 at 40 mg/kg/adm.

Animals from group 4 received one IP injection of MOR208 at 11 mg/kg/inj on D23, D27, D30, D34, D37 and D41 followed by a single IP injection of MOR208 at 11 mg/kg/inj on D48 (twice a week).

Animals from group 5 received daily PO administration of ABT-199 at 20 mg/kg/adm in combination with IP injections of MOR208 at 11 mg/kg/inj on D23, D27, D30, D34, D37 and D41 and on D48, D51, D55 and D58.

Animals from group 6 received daily PO administration of ABT-199 at 40 mg/kg/adm in combination with two cycles of one IP injection of MOR208 at 11 mg/kg/inj on D23, D27, D30, D34, D37 and D41 and on D48, D51, D55 and D58.

All study data, including animal bodyweight measurements, clinical and mortality records, and treatment were scheduled and recorded on Vivo Manager® database (Biosystemes, Dijon, France). The viability and behavior were recorded every day. Body weights and tumor volume were measured twice a week. The length and width of the tumor were measured twice a week with calipers and the volume of the tumor was estimated by the formula:

$$Tumor volu me = \frac{{width}^{2} \times length}{2}$$

### Results

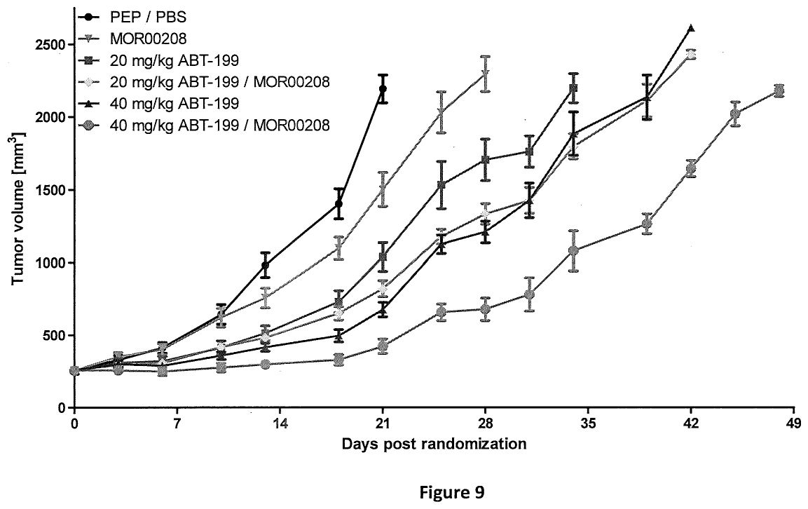

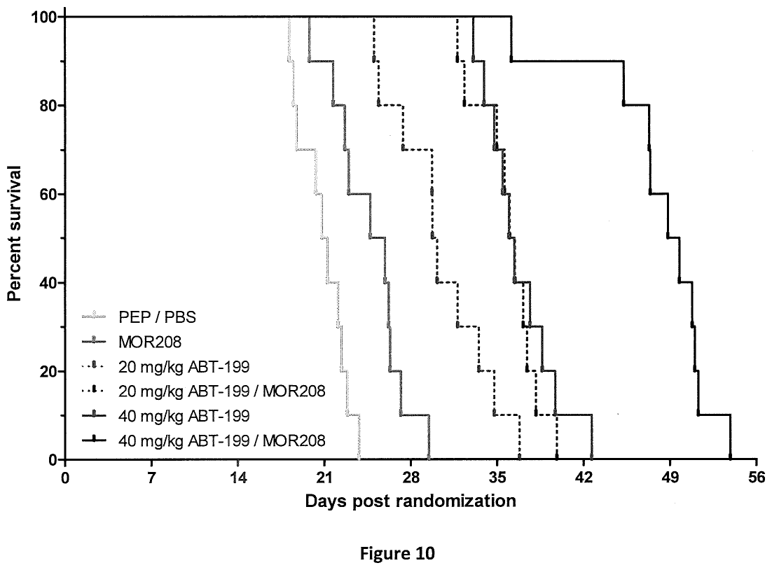

The combination treatment of MOR00208 and ABT-199 at a dose of 20 and 40 mg/kg resulted in a superior and statistically significant inhibition of the growth of the Toledo lymphoma cells compared to the vehicle control and both monotherapy regimens. The anti-tumor effect of MOR00208 in combination with ABT-199 shows even more pronounced when the survival of the mice from randomization until the humane endpoint (tumor volume of 2000 mm³) is analyzed. The median survival and the respective increased life span of the combination groups were superior compared to the respective monotherapies. A further characterisation of the combination effect categorizes the result of the combination of MOR00208 as potentiation since the effect of the combination is bigger than the sum of the repesctive monotherapies (FIG. 9, Table 7, FIG. 10, Table 8).

TABLE 7
Effect of MOR00208 and ABT-199 alone and in combination
on median survival in a s.c. Toledo SCID mouse model.

| | | Median | Median % | Evaluation |
|---|---|---|---|---|
| | | Tumor | Increase in | of combi- |
| | | Doubling | Doubling | natorial |
| Group | Treatment | Time (Days) | Time (IDT)^{§} | effects |
| 1 | PEP/PBS | 6.8 | n.a. | n.a. |
| 4 | MOR00208 11 mg/kg | 8.0 | 18 | n.a. |
| 2 | ABT-199 20 mg/kg | 9.6^{a} | 44 | n.a. |
| 3 | ABT-199 40 mg/kg | 11.1^{a} | 64 | n.a. |
| 5 | ABT-199 20 mg/kg & | 11.5^{b} | 70 | Synergy^{c} |
| | MOR00208 11 mg/kg | | | |
| 6 | ABT-199 40 mg/kg & | 13.5^{b} | 100 | Synergy^{d} |
| | MOR00208 11 mg/kg | | | |

^{§}vs. PEP/PBS Control,
^{a}significantly different to PBS Control,
^{b} significantly different to vehicle control and respective monotherapy groups,
^{c}Synergy vs. the respective monotherapy groups as IDT Combination ABT-199 20 mg/kg & MOR00208 11 mg/kg (70%) > sum of the effects of the respective monotherapy groups IDT ABT-199 20 mg/kg (44%) + IDT MOR00208 11 mg/kg (18%) = 62%.
^{d}Synergy vs. the respective monotherapy groups as IDT Combination ABT-199 40 mg/kg & MOR00208 11 mg/kg (100%) > sum of the effects of the respective monotherapy groups IDT ABT-199 40 mg/kg (64%) + IDT MOR00208 11 mg/kg (18%) = 82%. (

TABLE 8
Effect of MOR00208 and ABT-199 alone and in combination
on median survival in a s.c. Toledo SCID mouse model.

| | | Median | | |
|---|---|---|---|---|
| | | Survival | Median % | Evaluation |
| | | (Days Post- | Increase in | of combi- |
| | | Random- | Lifespan | natorial |
| Group | Treatment | ization) | (ILS)§ | effects |
| 1 | PEP/PBS | 21.0 | n.a. | n.a. |
| 4 | MOR00208 11 mg/kg | 25.3 | 20 | n.a. |
| 2 | ABT-199 20 mg/kg | 30.0^{a} | 43 | n.a. |
| 3 | ABT-199 40 mg/kg | 36.2^{a} | 72 | n.a. |
| 5 | ABT-199 20 mg/kg & | 36.2 ^{b} | 79 | Synergy^{c} |
| | MOR00208 11 mg/kg | | | |
| 6 | ABT-199 40 mg/kg & | 49.3 ^{b} | 134 | Synergy^{d} |
| | MOR00208 11 mg/kg | | | |

§vs. PEP/PBS Control,
^{a}significantly different to PBS Control,
^{b} significantly different to vehicle control and respective monotherapy groups,
^{c}Synergy vs. the respective monotherapy groups as ILS Combination ABT-199 20 mg/kg & MOR00208 11 mg/kg (72%) > sum of the effects of the respective monotherapy groups ILS ABT-199 20 mg/kg (43%) + ILS MOR00208 11 mg/kg (20%) = 63%.
^{d}Synergy vs. the respective monotherapy groups as ILS Combination ABT-199 40 mg/kg & MOR00208 11 mg/kg (134%) > sum of the effects of the respective monotherapy groups ILS ABT-199 40 mg/kg (72%) + ILS MOR00208 11 mg/kg (20%) = 92%.

1.-12. (canceled)

13. A method for treatment of chronic lymphocytic leukemia, acute lymphoblastic leukemia, or a non-Hodgkin's lymphoma in a human subject in need thereof, the method comprising administering to the human subject a therapeutically effective amount of a combination comprising an antibody specific for CD19 and a BCL-2 inhibitor, wherein the antibody specific for CD19 comprises an HCDR1 region comprising sequence SYVMH (SEQ ID NO: 1), an HCDR2 region comprising sequence NPYNDG (SEQ ID NO:2), an HCDR3 region comprising sequence GTYYYGTRVFDY (SEQ ID NO:3), an LCDR1 region comprising sequence RSSKSLQNVNGNTYLY (SEQ ID NO:4), an LCDR2 region comprising sequence RMSNLNS (SEQ ID NO:5), and an LCDR3 region comprising sequence MQHLEYPIT (SEQ ID NO: 6).

14. The method according to claim 13, wherein the antibody specific for CD19 and the BCL-2 inhibitor are administered separately.

15. The method according to claim 13, wherein the BCL-2 inhibitor is administered prior to administration of the antibody specific for CD19.

16. The method according to claim 13, wherein the antibody specific for CD19 and the BCL-2 inhibitor are administered simultaneously.

17. The method according to claim 13, wherein the antibody specific for CD19 and the BCL-2 inhibitor are administered at a time where both drugs are active in the subject at the same time.

18. The method according to claim 13, wherein the human subject has chronic lymphocytic leukemia.

19. The method according to claim 13, wherein the human subject has acute lymphoblastic leukemia.

20. The method according to claim 13, wherein the human subject has a non-Hodgkin's lymphoma.

21. The method of claim 20, wherein the non-Hodgkin's lymphoma is selected from the group consisting of follicular lymphoma, small lymphocytic lymphoma, mucosa-associated lymphoid tissue lymphoma, marginal zone lymphoma, diffuse large B cell lymphoma, Burkitt's lymphoma, and mantle cell lymphoma.

22. The method according to claim 13, wherein the antibody specific for CD19 comprises a heavy chain variable region comprising the sequence EVOLVESGGGLVKPGGSLKLSCAASGYTFTSYVMHWVRQAPGKGLEWIGYINPYNDGT KYNEKFQGRVTISSDKSISTAYMELSSLRSEDTAMYYCARGTYYYGTRVFDYWGQGTL VTVSS (SEQ ID NO:10) and a light chain variable region comprising the sequence
(SEQ ID NO: 11)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIK.

23. The method according to claim 22, wherein the antibody specific for CD19 comprises a heavy chain constant domain comprising the sequence ASTKGPSVFPLAPSSKSTSGGTAALGCLVKDYFPEPVTVSWNSGALTSGVHTFPAVLQSS GLYSLSSVVTVPSSSLGTQTYICNVNHKPSNTKVDKKVEPKSCDKTHTCPPCPAPELLGG PDVFLFPPKPKDTLMISRTPEVTCVVVDVSHEDPEVQFNWYVDGVEVHNAKTKPREEQF NSTFRVVSVLTVVHQDWLNGKEYKCKVSNKALPAPEEKTISKTKGQPREPQVYTLPPSR EEMTKNQVSLTCLVKGFYPSDIAVEWESNGQPENNYKTTPPMLDSDGSFFLYSKLTVDK SRWQQGNVFSCSVMHEALHNHYTQKSLSLSPGK (SEQ ID NO:12) and a light chain constant domain comprising the sequence
(SEQ ID NO: 13)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.

24. The method according to claim 18, wherein the antibody specific for CD19 comprises a heavy chain variable region comprising the sequence EVOLVESGGGLVKPGGSLKLSCAASGYTFTSYVMHWVRQAPGKGLEWIGYINPYNDGT KYNEKFQGRVTISSDKSISTAYMELSSLRSEDTAMYYCARGTYYYGTRVFDYWGQGTL VTVSS (SEQ ID NO:10) and a light chain variable region comprising the sequence
(SEQ ID NO: 11)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIK.

25. The method according to claim 24, wherein the antibody specific for CD19 comprises a heavy chain constant domain comprising the sequence ASTKGPSVFPLAPSSKSTSGGTAALGCLVKDYFPEPVTVSWNSGALTSGVHTFPAVLQSS GLYSLSSVVTVPSSSLGTQTYICNVNHKPSNTKVDKKVEPKSCDKTHTCPPCPAPELLGG PDVFLFPPKPKDTLMISRTPEVTCVVVDVSHEDPEVQFNWYVDGVEVHNAKTKPREEQF NSTFRVVSVLTVVHQDWLNGKEYKCKVSNKALPAPEEKTISKTKGQPREPQVYTLPPSR EEMTKNQVSLTCLVKGFYPSDIAVEWESNGQPENNYKTTPPMLDSDGSFFLYSKLTVDK SRWQQGNVFSCSVMHEALHNHYTQKSLSLSPGK (SEQ ID NO:12) and a light chain constant domain comprising the sequence
(SEQ ID NO: 13)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.

26. The method according to claim 19, wherein the antibody specific for CD19 comprises a heavy chain variable region comprising the sequence EVOLVESGGGLVKPGGSLKLSCAASGYTFTSYVMHWVRQAPGKGLEWIGYINPYNDGT KYNEKFQGRVTISSDKSISTAYMELSSLRSEDTAMYYCARGTYYYGTRVFDYWGQGTL VTVSS (SEQ ID NO:10) and a light chain variable region comprising the sequence
(SEQ ID NO: 11)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIK.

27. The method according to claim 26, wherein the antibody specific for CD19 comprises a heavy chain constant domain comprising the sequence ASTKGPSVFPLAPSSKSTSGGTAALGCLVKDYFPEPVTVSWNSGALTSGVHTFPAVLQSS GLYSLSSVVTVPSSSLGTQTYICNVNHKPSNTKVDKKVEPKSCDKTHTCPPCPAPELLGG PDVFLFPPKPKDTLMISRTPEVTCVVVDVSHEDPEVQFNWYVDGVEVHNAKTKPREEQF NSTFRVVSVLTVVHQDWLNGKEYKCKVSNKALPAPEEKTISKTKGQPREPQVYTLPPSR EEMTKNQVSLTCLVKGFYPSDIAVEWESNGQPENNYKTTPPMLDSDGSFFLYSKLTVDK SRWQQGNVFSCSVMHEALHNHYTQKSLSLSPGK (SEQ ID NO:12) and a light chain constant domain comprising the sequence
(SEQ ID NO: 13)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.

28. The method according to claim 20, wherein the antibody specific for CD19 comprises a heavy chain variable region comprising the sequence EVQLVESGGGLVKPGGSLKLSCAASGYTFTSYVMHWVRQAPGKGLEWIGYINPYNDGT KYNEKFQGRVTISSDKSISTAYMELSSLRSEDTAMYYCARGTYYYGTRVFDYWGQGTL VTVSS (SEQ ID NO:10) and a light chain variable region comprising the sequence
(SEQ ID NO: 11)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIK.

29. The method according to claim 28, wherein the antibody specific for CD19 comprises a heavy chain constant domain comprising the sequence ASTKGPSVFPLAPSSKSTSGGTAALGCLVKDYFPEPVTVSWNSGALTSGVHTFPAVLQSS GLYSLSSVVTVPSSSLGTQTYICNVNHKPSNTKVDKKVEPKSCDKTHTCPPCPAPELLGG PDVFLFPPKPKDTLMISRTPEVTCVVVDVSHEDPEVQFNWYVDGVEVHNAKTKPREEQF NSTFRVVSVLTVVHQDWLNGKEYKCKVSNKALPAPEEKTISKTKGQPREPQVYTLPPSR EEMTKNQVSLTCLVKGFYPSDIAVEWESNGQPENNYKTTPPMLDSDGSFFLYSKLTVDK SRWQQGNVFSCSVMHEALHNHYTQKSLSLSPGK (SEQ ID NO:12) and a light chain constant domain comprising the sequence
(SEQ ID NO: 13)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.
